(19) United States
(12) Patent Application Publication
(10) Pub. No.: US 2011/0104213 A1
(43) Pub. Date: May 5, 2011

(54) Porous Biomolecule-Containing Metal-Organic Frameworks

(75) Inventors: Nathaniel Louis Rosi, Pittsburgh, PA (US); Jihyun An, Evanston, IL (US)

(73) Assignee: University of Pittsburgh-Of the Commonwealth System of Higher Education, Pittsburgh, PA (US)

(21) Appl. No.: 12/915,178

(22) Filed: Oct. 29, 2010

Related U.S. Application Data
(60) Provisional application No. 61/256,487, filed on Oct. 30, 2009.

(51) Int. Cl. B01J 20/22; C07F 3/06; C01B 31/20; A61K 31/166; A61K 9/00; A61P 9/06

(52) U.S. Cl. 424/400; 544/226; 502/401; 423/437.1; 514/619; 428/219

(57) ABSTRACT

The present invention relates to compositions including porous biomolecule-containing metal-organic frameworks and methods for their preparation. The porous biomolecule-containing metal-organic frameworks can include a metal component and a biomolecule component. The pores located within the frameworks have a pore space and said pore space is capable to adsorb materials therein. These compositions of the present invention are useful in a wide variety of applications, such as, but not limited to, hydrogen and carbon dioxide sequestration, separation and storage; carbon dioxide uptake; and drug storage and release.

### CROSS-REFERENCE TO RELATED APPLICATION

This application claims the benefit of U.S. Provisional Patent Application Ser. No. 61/256,487, filed Oct. 30, 2009, and entitled “Porous Biomolecule-Containing Metal-Organic Frameworks,” which is herein incorporated by reference in its entirety.

### BACKGROUND OF THE INVENTION

1. Field of the Invention

The present invention relates to compositions including porous biomolecule-containing metal-organic frameworks; porous, biomolecule-containing metal-organic macrocyclic structures; and the like; and methods for their preparation. These compositions are useful in a wide variety of applications, in particular, hydrogen and carbon dioxide capture, separation and storage; carbon dioxide uptake; and drug storage and release.

2. Background Information

The metal-organic frameworks (“MOFs”) known in the art are crystalline compounds which consist of inorganic secondary building units (e.g. metal clusters or metal ions) linked together with organic molecules to form one-, two-, or three-dimensional structures. MOFs are also known as hybrid frameworks and coordination polymers. MOFs are used in applications such as gas purification, gas separation, catalysis and sensors. MOF-5 is a known matrix which is built up by Zn₄O groups on the corners of a cubic lattice, connected by 1,4-benzenedicarboxylic acid. MOF-74 is a known matrix composed mainly of carbon and zinc.

Frameworks can be prepared by reflux, precipitation, and recrystallization processes. For example, reagents can be sealed in an autoclave with water or solvent and heated to a temperature of from 100° C. to 250° C. This allows for the activation energy needed to assemble the complex framework structures to be achieved without the solvent evaporating.

It would be desirous for the metal-organic frameworks to have pores and/or channels structured therein such that gas and/or other material may be adsorbed or trapped within the spaces. These porous materials constructed from metal ions and organic molecular building blocks can be used for a variety of applications. For example, for clean energy applications, porous metal-organic structures can be used to adsorb and trap large amounts of carbon dioxide (CO₂) and hydrogen (H₂) gases; for drug delivery applications, porous metal-organic structures can be used to store and release drug molecules such that the drug can be administered in a controlled manner over a period of time. The properties of such materials can be adjusted based on the selection of the metal ions and the molecular building blocks.

### BRIEF DESCRIPTION OF THE DRAWINGS

FIG. 1 is a diagram showing the chemical structure of a zinc-adeninate macrocycle, in accordance with an embodiment of the present invention.

FIG. 2 is a diagram showing zinc-adeninate building units connected by biphenyldicarboxylate ligands which exhibit void spaces formed therein, in accordance with an embodiment of the present invention.

FIG. 3 is diagram showing the three-dimensional framework of a cobalt-adeninate metal-organic framework which exhibits pores that are densely lined with pyrimidal nitrogens and amino groups from the adenine building blocks, in accordance with an embodiment of the present invention.

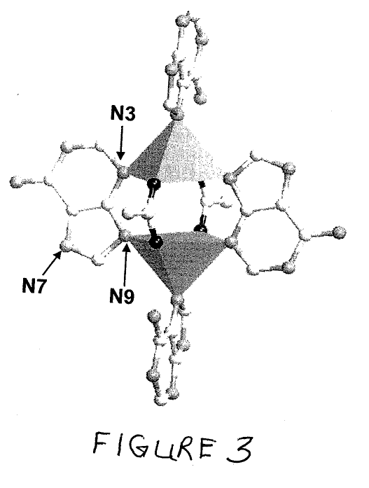

### SUMMARY OF THE INVENTION

In an aspect, the present invention provides a composition including a biomolecule-containing metal-organic framework having pores structured therein, said framework including a metal component and a biomolecule component, said pores capable to adsorb material therein. The metal component can be selected from the group consisting of metal cluster, metal ion or combination thereof. The metal component can be selected from the group consisting of Cu, Ti, Cr, Fe, Ni, Mn, Co, Zn, GaN, BN, Si₃N₄, TiN, TiC, TiB₂ GaAs, GaP, InP, AlP, and salts and mixtures thereof. The biomolecule can include a nucleobase. The nucleobase can be selected from the group consisting of adenine, guanine, cytosine, thymine, uracil and mixtures thereof. The biomolecule can be biologically compatible with the metal component.

The aforementioned composition can further include an organic component. The organic component can include an organic ligand. The organic ligand can include a multitopic material. The organic ligand can be selected from the group consisting of dicarboxylate ligands, tricarboxylate ligands, tetracarboxylate ligands, other multicarboxylated ligands, dipyridyl ligands, tripyridyl ligands, tetrapyridyl ligands, other multipyridal ligands, dicyano ligands, tricyano ligands, tetracyano ligands, other multicyano ligands, diphosphonate ligands, triphosphonate ligands, tetraphosphonate ligands, other multiphosphonate ligands, dihydroxyl ligands, trihydroxyl ligands, tetrahydroxyl ligands, other multihydroxyl ligands, disulfonate ligands, trisulfonate ligands, tetrasulfonate ligands, other multisulfonate ligands, diimidazolate ligands, triimidazolate ligands, tetraimidazolate ligands, other multiimidazolate, ligands, ditriazolate ligands, tritriazolate ligands, tetratriazolate ligands, other multitriazolate ligands, and mixtures and combinations thereof.

The framework can be selected from the group of structures consisting of macrocyclic structures, crystalline structures, polyhedra, extended framework structures, and combinations thereof. The framework can include a plurality of units connected by a linker. The linker can be selected from 1,4-benzene dicarboxylate; 2,6-naphthalene dicarboxylate; 4,4′-biphenyl dicarboxylate; 4,4″-terphenyl dicarboxylate; 4,4′-[(2,5-dimethoxy-1,4-phenylene)di-2,1-ethenediyl]bis-benzoic acid; 1,3,5-benzene tricarboxylate; 4,4′,4,4″-benzene-1,3,5-triyl-tribenzoate; 4,4′,4″-[benzene-1,3,5-triyl-tris(ethyne-2,1-diyl)]tribenzoate; 4,4′,4,4″-[benzene-1,3,5-triyl-tris(benzene-4,1-diyl)]tribenzoate; and mixtures thereof.

In one embodiment, the metal component includes zinc salt. In another embodiment, the nucleobase includes adenine.

The pores can be capable to adsorb material selected from the group consisting of gas, drug, protein, polymer and combinations thereof. The gas can be selected from the group consisting of carbon dioxide, hydrogen, nitrogen, and mixtures thereof. The material can be a drug and the framework can be capable of a controlled release of the drug from the pores. The pores can be modified by cation exchange of cations in the pores with different cations.

The biomolecule-containing metal-organic framework can have a BET surface area from about 1,000 to about 4,000 m²/g. Further, the biomolecule-containing metal-organic framework can have a pore volume from about 2 to about 6 cm³/g or about 4.2 cm³/g.

In another aspect, the present invention provides a method for preparing a biomolecule-containing metal-organic framework having pores structured therein, the pores capable to adsorb materials therein, the method including reacting a metal component and a biomolecule component.

In another aspect, the present invention provides a method for storing and controllably releasing drug material including the aforementioned composition.

In still another aspect, the present invention provides a method for capturing and storing carbon dioxide material including the aforementioned composition.

### DETAILED DESCRIPTION OF THE INVENTION

The composition of the present invention includes biomolecule-containing porous metal-organic frameworks composed of metal and biomolecule building units. As used herein and the claims, the term “porous metal-organic framework” includes biomolecule-containing porous metal-organic macrocycles, macrocyclic structures, crystalline structures, polyhedra and extended framework structures. The porous metal-organic framework includes a metal component and a biomolecule component. The metal component includes a metal cluster, a metal ion, or combinations thereof. Non-limiting examples of suitable metal components include Cu, Ti, Cr, Fe, Ni, Mn, Co, Zn, GaN, BN, Si₃N₄, TiN, TiC, TiB₂ GaAs, GaP, InP, AlP, and salts and mixtures thereof. In one embodiment, the metal component includes zinc (Zn) ions. The biomolecule component includes a nucleobase. Non-limiting examples of suitable nucleobases include, but are not limited to, adenine, guanine, cytosine, thymine, uracil and mixtures thereof. The metal component and biomolecule component can be biologically compatible such that they can be used as building blocks for the construction of discrete metal-biomolecule macrocycles, macrocyclic structures, crystalline structures, polyhedra, and extended framework structures. In one embodiment, the framework can include more than one macrocycle. In a further embodiment, the macrocyles can be connected by hydrogen bonds.

The porous metal-organic framework of the present invention can optionally include an organic component. The optional organic component includes an organic ligand. The organic ligand is multitopic, such as, but not limited to, ditopic, tritopic, and tetratopic. As used herein, the term “topicity” refers to the number of functional groups present that can bind a metal. For example, a ditopic ligand has two functional groups capable of binding a metal, a tritopic ligand has three functional groups and a tetratopic ligand has four functional groups. Non-limiting examples of suitable organic ligands for use in the present invention include dicarboxylate ligands, tricarboxylate ligands, tetracarboxylate ligands, other multicarboxylated ligands, dipyridyl ligands, tripyridyl ligands, tetrapyridyl ligands, other multipyridal ligands, dicyano ligands, tricyano ligands, tetracyano ligands, other multicyano ligands, diphosphonate ligands, triphosphonate ligands, tetraphosphonate ligands, other multiphosphonate ligands, dihydroxyl ligands, trihydroxyl ligands, tetrahydroxyl ligands, other multihydroxyl ligands, disulfonate ligands, trisulfonate ligands, tetrasulfonate ligands, other multisulfonate ligands, diimidazolate ligands, triimidazolate ligands, tetraimidazolate ligands, other multiimidazolate, ligands, ditriazolate ligands, tritriazolate ligands, tetratriazolate ligands, other multitriazolate ligands, and mixtures or combinations thereof.

In one embodiment, the metal component is zinc salt. In another embodiment, the nucleobase is adenine.

The present invention also provides a method for preparing a porous metal-organic framework. The method includes reacting the metal component, the biomolecule component, and the optional organic component.

The biomolecule-containing porous metal-organic frameworks of the present invention can be of various shapes and sizes. The frameworks can be one-, two-, or three-dimensional structures. The frameworks can be anionic. Moreover, the porous metal-organic frameworks exhibit pores and/or channels throughout the structure. The inside of the pores and/or channels can be cationic. A variety of cations can be present within the pores, such as, but not limited to, dimethylammonium. The size, shape, volume and number of pores and/or channels can vary. The space or volume within the pores and/or channels is useful to adsorb and store material. The material can include gas (e.g., molecules), such as, but not limited to, hydrogen, carbon dioxide, nitrogen, and mixtures thereof; drug (e.g., molecules); proteins; polymers; and mixtures or combinations thereof. The stored material in the pore space or volume can then be released in a controlled manner from the frameworks.

The size of the pores can be controlled by cation exchange. Further, the functionality of the pores, such as, the capability to adsorb gas or other materials, also can be controlled by cation exchange. As used herein, the term “cation exchange” refers to replacing cations located in the pores with different cations. For example, the cation exchange can include the exchange or replacement of cations located inside the pores, such as, but not limited to, dimethylammonium cations with different cations, such as, but not limited to, tetramethylammonium cations, tetramethylammonium cations, tetraethylammonium cations, tetrabutylammonium cations, and mixtures thereof. The tetramethylammonium cations, tetraethylammonium cations and tetrabutylammonium cations are slightly larger in size than the dimethylammonium cations. The larger cations will occupy a greater amount of space within the pore and therefore, the remaining (e.g., open) pore volume can be reduced. In an alternate embodiment, wherein the cations located within the pores are exchanged with cations having a smaller size, the smaller cations will occupy a lesser amount of space within the pore and therefore, the remaining (e.g., open) pore volume can be increased. Thus, the exchange of cations located inside the pores with different cations (e.g., larger or smaller) allows for the pore volume to be modified (e.g., tuned) in accordance therewith. In alternate embodiments, wherein the pore volume is increased, the pore volume can be about 4.2 cm³/g or higher. Further, without intending to be bound by any particular theory, it is believed that the introduction of slightly larger cations can modify (e.g., increase) the pore space to allow the pore to accommodate a greater amount of material therein. Furthermore, the BET surface area can be modified (e.g., tuned) as a result of cation exchange within the pores. For example, depending on the different cations used as replacements for the cations located inside the pores, the BET surface area may increase or decrease. In alternate embodiments, wherein the BET surface area is increased, the BET surface area can be about 4000 m²/g or higher.

In one embodiment, wherein the metal component is zinc salt, the biomolecule component is adenine, and the organic component is a dicarboxylate ligand, the framework can include zinc-adeninate octahedral units connected with a biphenyldicarboxylate linker to form a three-dimensional structure that can be either microporous or mesoporous. The framework can include cobalt-adeninate building units and can have Lewis basic sites (amino and pyrimidil) from the adenine exposed to the pores. In alternate embodiments, the linker can be selected from a variety of materials known in the art. The linkers can include linear linkers, trigonal linkers and mixtures thereof. Non-limiting examples of linear linkers include, but are not limited to, 1,4-benzene dicarboxylate; 2,6-naphthalene dicarboxylate; 4,4′-biphenyl dicarboxylate; 4,4″-terphenyl dicarboxylate; 4,4′-[(2,5-dimethoxy-1,4-phenylene)di-2,1-ethenediyl]bis-benzoic acid, and mixtures thereof. Non-limiting examples of trigonal linkers include, but are not limited to, 1,3,5-benzene tricarboxylate; 4,4′,4,4″-benzene-1,3,5-triyl-tribenzoate; 4,4′,4″-[benzene-1,3,5-triyl-tris(ethyne-2,1-diyl)]tribenzoate; 4,4′,4,4″-[benzene-1,3,5-triyl-tris(benzene-4,1-diyl)]tribenzoate, and mixtures thereof. Without intending to be bound by any particular theory, it is believed that increasing the length and/or size of the linker used to connect units within the framework, can result in an increase in pore volume. For example, the use of terphenyl dicarboxylate can produce a mesoporous framework having a higher pore volume than the pore volume of a mesoporous framework using biphenyl dicarboxylate linker. In alternate embodiments, a mesoporous framework in accordance with the present invention can include a pore volume of from about 2 to about 4.2 cm³/g or from about 4.2 to about 6 cm³/g or from about 2 to about 6 cm³/g. In one embodiment, the pore volume is about 4.2 cm³/g.

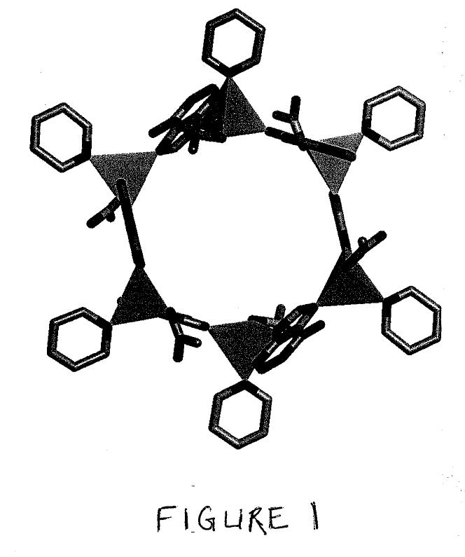

In one embodiment, a porous metal-organic framework in accordance with the present invention is formed using single crystal growth methods. For example, the metal compound, such as zinc salt, is reacted with the nucleobase, such as adenine, at room temperature in the presence of solvent (such as, dimethylformamide (DMF), pyridine and mixtures thereof), under solvothermal conditions (such as, heating) to produce discrete zinc-adeninate macrocycles. Adeninate can coordinate metal ions through any of its five nitrogens. The macrocycles can be hexameric (e.g., containing six subunits or moieties), as shown in FIG. 1. The hexameric macrocyle, in FIG. 1, contains six zinc tetrahedra, each at a corner of the hexagon, and six adeninate ligands, at the edges of the hexagon which bridge the zinc tetrahedra together through imidazole nitrogens. The remaining two coordination sites on the zinc are occupied by pyridine and acetate. For example, six Zn²⁺ occupy the vertices of the macrocycle and adeninates bridge the Zn²⁺ through their imidazolate nitrogens. Each Zn²⁺ binds in a tetrahedral fashion to two adeninates, one pyridine molecule, and one dimethylcarbamate anion (formed in-situ).

A plurality of discrete macrocycles can self-assemble (e.g., stack together) through hydrogen bonding interactions into a three-dimensional crystalline structure. This structure can consist of alternating layers of macrocycles that stack in an a-b-c fashion. Each macrocycle forms a total of 12 hydrogen bonds (two per adeninate) with its six nearest-neighbor macrocycles within the structure. This packing motif (e.g., crystalline stacking) results in the formation of channels, such as pores and/or cylindrical cavities, arranged periodically throughout the three-dimensional structure. The confines of each cavity/pore are defined by one central macrocycle and fragments of the six nearest-neighbor macrocycles. In one embodiment, the three-dimensional crystalline structure includes one-dimensional cavities/pores running along the c-crystallographic direction. Within the cavities/pores can reside dimethylammonium cations (the product of DMF (solvent) decomposition). The size, shape and number of the cavities/pores can vary. In one embodiment, the pores are approximately 6 angstroms in diameter. The unit cell parameters for the crystal structure are: R-3 a=b=17.74072 c=33.450; alpha=beta=90 gamma=120; cell volume=9117.3.) The molecular formula for the materials shown in FIG. 1 is as follows:

Zn₆(ad)₆(py)₆(ac)₆ and Zn₆(ad)₆(py)₆(cb)₆ (I)

wherein “Zn” represents zinc, “ad” represents adeninate, “ac” represents acetate, “py” represents pyridine, and “cb” represents carbamate.

In another embodiment, the present invention includes materials having the following molecular formula:

Zn₈(ad)₄(BPDC)₆O′2NH₂(CH₃)₂, 11H₂O, 8DMF (II)

wherein “Zn” represents zinc, “ad” represents adeninate, “BPDC” represents 4,4-biphenyldicarboxylate, and “DMF” represents dimethylformamide.

The material having the molecular formula of (II) above is constructed from one-dimensional columns consisting of chains of corner-sharing zinc-adeninate octahedra. As shown in FIG. 4, these columns are linked together into a crystalline three-dimensional porous framework using 4,4′-biphenyldicarboxylate linkers. This framework consists of metal-organic secondary building units (the zinc-adeninate octahedra). These units are then connected together with a biphenyldicarboxylate linker. This three-dimensional bio-structure contains large one-dimensional channels oriented along the c-crystallographic direction. Thus, the method of the present invention can produce porous metal-organic frameworks having hierarchical porosity; e.g., pores within zinc adeninate octahedra and pores generated by linking the octahedra together with the biphenyldicarboxylate units. The unit cell parameters for the crystal structure are: I4(1)22 a=b=38.2372 c=11.1753; alpha=beta=gamma=90.

In an embodiment, the material having the molecular formula of (II) was subjected to cation exchange. The dimethylammonium cations located within the pores were exchanged with (i) tetramethylammonium cations, (ii) tetraethylammonium cations and (iii) tetrabutylammonium cations. Prior to the cation exchange, wherein the pores contained dimethylammonium cations, the pore volume was measured as 0.75 cc/g and the BET was measured as 1680 m²/g. When the dimethylammonium cations were exchanged with tetramethylammonium cations, the pore volume and BET were measured as 0.65 cc/g and 1460 m²/g, respectively. When the dimethylammonium cations were exchanged with tetraethylammonium cations, the pore volume and BET were measured as 0.55 cc/g and 1220 m²/g, respectively. When the dimethylammonium cations were exchanged with tetrabutylammonium cations, the pore volume and BET were measured as 0.37 cc/g and 830 m²/g, respectively.

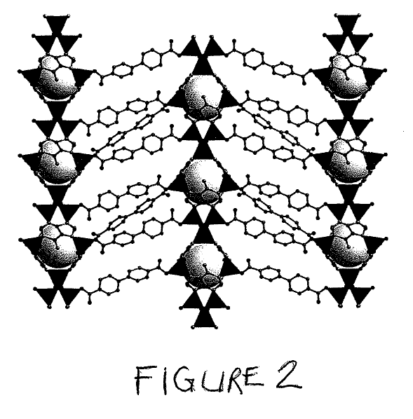

FIG. 2 shows a framework in accordance with an embodiment of the present invention wherein zinc-adeninate columnar building units are connected by biphenyldicarboxylate ligands within a crystal structure. The spheres indicate void (e.g., pore) space within the zinc-adeninate octahedra which form the zinc-adeninate columns.

In another embodiment, the present invention includes materials having the following molecular formula:

Co₂(ad)₂(CO₂CH₃)₂.2DMF, 0.5H₂O (III)

wherein “Co” represents cobalt, “ad” represents adeninate, and “DMF” represents dimethylformamide.

In one embodiment, the material having molecular formula (III) is prepared via a solvothermal reaction between cobalt acetate tetrahydrate and adenine in N,N-dimethylformamide. As shown in FIG. 5, this material is constructed from cobalt-adeninate-acetate “paddle-wheel” clusters in which two Co²⁺ are each bridged by two adeninates via the N3 and N9 positions and two acetates in the dimonodentate alignment. These units are linked together through apical coordination of the adeninate N7 to Co²⁺ on neighboring clusters to generate a three-dimensional framework structure. There are open cavities/pores periodically distributed throughout the structure. The pores are densely lined with Lewis basic amino and pyrimidine groups, because the connectivity of the framework leaves these groups uncoordinated and exposed to the channels. The unit cell parameters for the crystal structure are: I41/A a=b=15.4355 c=22.7753; alpha=beta=gamma=90.

In another embodiment, the present invention includes materials having the following molecular formula:

Zn₈(ad)₄(O)₂(BPDC)₆.4Me₂ NH₂, xDMF, xH₂O (IV)

wherein “Zn” represents zinc, “ad” represents adeninate, “BPDC” represents 4,4-biphenyldicarboxylate, “Me” represents methyl, “DMF” represents dimethyl formamide, and “x” represents the number of molecules in the pore.

The material having the molecular formula of (IV) above is constructed from discrete zinc-adeninate octahedral cages. Three biphenyldicarboxylates coordinate to the Zn²⁺ tetrahedra on each open face of the octahedral cage and connect the octahedra together into a diamond-like network, where each zinc-adeninate octahedral cage serves as a tetrahedral four-connected building unit. Due to the length of the dicarboxylate linker and the size of the zinc-adeninate building unit, the resulting framework exhibits large mesoporous pores or cavities. This framework is also anionic and contains dimethylammonium cations within its pores. The unit cell parameters for the crystal structure are: IA-3D a=b=c=69.1153; alpha=beta=gamma=90.

The solid-state crystalline structure of the zinc-adeninate macrocycles contains cylindrical openings, such as pores, channels, or cavities which are about 22 angstroms in length and about 6 angstroms in diameter. The aperture to these cavities is small, however, measuring only about 1-2 angstroms. The pyridine ligands constrict the entrance to these cavities. Without intending to be bound by any theory, it is believed that heating the materials may remove some of the pyridine ligands, thereby introducing an access route to the cylindrical cavities and therefore enabling the cavities to store and trap materials, as previously described. For example, it is believed that the cavities can be used to store hydrogen at low pressures because when hydrogen is introduced into the pores of the framework, it remains there until significant external vacuum is applied to release it from the pores. Storing hydrogen at low pressures is desirable for various applications, including, but not limited to, on-board hydrogen fueling applications and economic storage of large amounts of hydrogen in laboratory cylinders. Furthermore, the biomolecule-containing porous metal-organic frameworks of the present invention could potentially be used for trapping CO₂. This is an important industrial application, in particular, for the removal of CO₂ from the emissions of various manufacturing or process facilities, such as, but not limited to, coal-fired power plants.

In one aspect of the present invention, the biomolecule-containing porous metal-organic frameworks of the present invention include anionic frameworks, and therefore, as previously mentioned, the cations located in the pores can be exchanged with other cations. For example, the zinc-adenine frameworks described above herein are anionic frameworks and therefore, the dimethylammonium cations present in the pores can be exchanged out. In general, cations are mobile and can be removed and replaced with a variety of other cationic molecules via simple cation exchange. In one embodiment, the cation can be used to balance the charge. Without intending to be bound by any particular theory, it is believed that the pore size and pore functionality of the framework can be modified by introducing different cations into the pores. For example, replacing at least a portion of the cations in the pores with larger cations can result in an increase in the capacity of the pores. Thus, the pores can adsorb a greater amount of materials, as previously described. In one embodiment, the dimethylammonium cations in the pores can be exchanged with tetramethylammonium cations (e.g., slightly larger cations). The introduction of these slightly larger cations can modify the pore space and allow the pore to accommodate a greater amount of material. In an embodiment, the larger pore space can accommodate up to 100 cm³/g of carbon dioxide. Further, it is believed that constriction of the pore space may allow for increased inter-sorbent interactions and therefore, a higher capacity for storage.

The cobalt-adenine frameworks described above exhibit Lewis basic groups exposed to the pore channels. These groups are optimal for interacting with CO₂. The CO₂ isotherm is also depicted as a reversible type I isotherm and adsorption and desorption cycles can be repeated with complete reversibility. At 1 bar, this material adsorbs 6.0 mmol/g at 273K and 4.1 mmol/g at 298K. However, its N₂ uptake at 273K and 298K showed only 0.43 mmol/g and 0.13 mmol/g, respectively, indicating that the pores preferentially adsorb CO₂ over N₂. The isoteric heat of CO₂ sorption were calculated from the Clausius-Clapeyron equation using adsorption data collected at 298K, 303K, 308K and 313K. The initial heat of adsorption is 45 kj/mol, which is one of the highest observed for MOFs and CO₂ in comparison to known values in the art.

In accordance with the present invention, the pore size, shape, and functionality of the porous metal-organic frameworks may be modified and controlled. Therefore, the frameworks can be used as a scaffold to produce a wide variety of functionally unique materials by selecting various cation species to be introduced into the framework. This capability is useful for a wide variety of applications, such as, but not limited to, gas storage, drug delivery (e.g., controlled release), carbon dioxide capture, and the like. Since the porous metal-organic frameworks include those constructed in part from biomolecular components, the frameworks can be particularly suitable for drug storage/release applications as compared to these frameworks in the prior art that may not be biologically compatible.

The materials of the present invention are stable in a variety of organic solvents, water, and biological buffers, such that they can be used in various types of environments. For example, the frameworks can remain crystalline in solvents, such as, but not limited to, water, acetonitrile, chloroform, ethanol and mixtures thereof. In another example, the frameworks can remain crystalline in biological buffers for several weeks or longer. Further, the materials of the present invention demonstrate thermal stability at a variety of temperature conditions. For example, in one embodiment, the frameworks can remain crystalline up to a temperature of 250° C. The frameworks are also essentially permanently porous with a BET surface area ranging from about 1000 to about 4000 m²/g or from about 2000 to about 4000 m²/g, based on gas sorption measurements.

In non-limiting embodiments, the chemistry of the pores can be modified for specific applications, such as CO₂ sequestration or gas and liquid separations.

In one embodiment, the present invention provides the ability to selectively assemble biological components into hierarchical structures using metal ions. Biomolecules can be used as building blocks for porous metal-organic crystalline materials. Biomolecules can be bio-compatible and recyclable. These characteristics are desirous for environmental applications (e.g., environmental cleanup/remediation), food industry applications (e.g., food fresheners/deodorizers), and biomedical applications (e.g., nitric oxide delivery, drug delivery, and enzyme sequestration). For example, in drug delivery applications, the porous metal-organic frameworks of the present invention remain stable throughout the duration of drug release and then they biodegrade after complete release of drug.

In one embodiment, wherein the porous biomolecule-containing metal-organic framework has been subjected to a cation exchange process and the pores are at least partially loaded with material (e.g., different material than the material that was originally in the pores when the framework was formed), the framework can be soaked in a buffer solution, such as, but not limited to, phosphate buffered saline. In this embodiment, the material which is at least partially loaded in the pores can include drug molecules for a drug delivery application.

Porous materials generally can be used as a mechanism for release agents, such as but not limited to drug molecules, because their pore size and functionality can be controlled and adjusted based on the release profile of particular drug molecules. Porous materials constructed from molecular building blocks and metal ions are particularly useful because 1) the pore metrics and functionality can be systematically tuned and 2) they can be constructed from biomolecules and biologically-compatible metal ions. Therefore, materials can be designed and created to be biodegradable and how they interact with and store drug molecules can be substantially controlled.

The porous biomolecule-containing metal-organic frameworks of the present invention demonstrate at least one of the following advantages: 1) high permanent porosity; 2) high stability under a wide variety of conditions, including biological and environmental media; 3) ability to vary pore functionality by performing facile cation-exchange experiments; 4) high affinity for CO₂ and 5) ability to maintain crystalline integrity throughout the release of material from pores therein, however, following complete release of material, ability to lose crystalline integrity and biodegrade.

### EXAMPLES

### Example 1

### Zn₆(ad)₆(py)₆(cb)₆, H₂ and CO₂ Sorption Studies

A mixture of adenine and zinc nitrate was dissolved in dimethylformamide (DMF) and pyridine. Heating this solution resulted in the formation of a solid-state crystalline structure of zinc-adeninate macrocycles having cylindrical cavities which were approximately 22 angstroms in length and 6 angstroms in diameter. The aperture to these cavities was small, measuring only approximately 1-2 angstroms. The Zn6(adeninate)6(py)6(cb)6 was heated (e.g., activated) to 125° C., some of the pyridine molecules were removed and gases then accessed the pores. This was evidenced by gas sorption studies performed on the material. The activated material adsorbed 2.19 weight percent H₂ at 77 K and 760 mmHg, which compared favorably to other similar porous molecular materials. Significant hysteresis was observed when we monitored H₂ desorption. Approximately 1% by weight H₂ remained trapped within the material at pressures as low as 50 ton. Thus, it is believed that this type of material may be suitable to store hydrogen at low pressures because once the hydrogen was introduced into the pores of the material, it remained there until significant external vacuum was applied to remove it.

The CO₂ sorption properties of this material were also tested. The CO₂ sorption isotherm showed similar hysteresis as demonstrated for H₂, although not as dramatic. Therefore, this material and others like it may be suitable for trapping CO₂.

### Example 2

### Zn₈(ad)₄(BPDC)₆O″2NH₂(CH₃)₂, 11H₂O, 8DMF: Cation Exchange and CO₂ Sorption Studies

A mixture of adenine and zinc acetate dihydrate in dimethylformamide (DMF) and biphenyl dicarboxylic acid was prepared, and an anionic structure was formed having molecular structure Zn₈(ad)₄(BPDC)₆O″2NH₂(CH₃)₂, 11H₂O, 8DMF. Elemental analysis (EA) and thermogravimetric analysis (TGA) demonstrated that dimethylammonium cations (the product of DMF decomposition) as well as DMF and water resided in the pores of the structure. The dimethylammonium cations were exchanged out of the material via cation exchange. The potential to modify the pore size and functionality by introducing different cations into the pores was evaluated. In particular, it was shown that by introducing larger organic cations into the pores, the CO₂ capacity of the pores was increased. The CO₂ uptake of the evacuated as-synthesized material which contains dimethylammonium cations within the pores was measured. It was found that this material adsorbed 77 cm³/g of CO₂ at 0° C. This capacity for CO₂ was favorable. To determine whether the capacity of this material for CO₂ could be controlled, the dimethylammonium cations in the pores were exchanged with tetramethylammonium cations. Introducing this slightly larger cation modified the pore space and allowed it to accommodate a larger amount of CO₂, up to 100 cm³/g. Without intending to be bound by any theory, it was believed that the constriction of the pore space may allowed for greater inter-sorbent interactions and thus a higher capacity for storage.

### Example 3

### Zn₈(ad)₄(BPDC)₆O″2NH₂(CH₃)₂, 11H₂O, 8DMF: Drug Storage and Release Studies

Zn₈(ad)₄(BPDC)₆O″2NH₂(CH₃)₂, 11 H₂O, 8DMF, as prepared in accordance with the description in Example 2, was evaluated for its capacity to store and release drug molecules. Zn₈(ad)₄(BPDC)₆O″2NH₂(CH₃)₂, 11 H₂O, 8DMF, being an anionic material, was loaded with cationic drug molecules by soaking the material in water to remove all the DMF molecules and then performing cation exchange experiments to replace the dimethylammonium cations with cationic drug molecules. The drug release was then controlled through cation exchange with metal ions such as Mg²⁺ or Na⁺, which are common in biological systems. The capture and release of procainamide, a cationic drug molecule which is a class IA antiarhythmic drug used for life-threatening or symptomatic ventricular arrhythmias, was evaluated. One problem with procainamide is that it rapidly metabolizes and it has an elimination half-life of approximately 3 hours. It was a good candidate for controlled release, as it allowed for protracted dosing and protection of the procainamide from degradation during the dosing period.

The water-exchanged material was soaked with aqueous solutions of procainamide-hydrochloric acid (HCl) to load the pores of the material with procainamide molecules. Complete loading was confirmed by TGA, EA, and gas sorption experiments. To evaluate the release profile of the adsorbed procainamide and whether its release could be controlled via cation exchange with metal cations present in biological buffers, the procainamide-exchanged material was soaked in 0.1 M phosphate-buffered saline (PBS) having a pH of 7.4. The release of procainamide was monitored via high-pressure liquid chromatography (HPLC). Steady procainamide release was observed over approximately a 20-hour period, and total release of the procainamide was completed after approximately 72 hours. The crystalline integrity of the framework was maintained throughout the release process, as evidenced by X-ray powder diffraction experiments. However, after all of the drug molecules had been released, the material began to lose its crystalline integrity and appeared to break down in the biological buffers. This feature may be useful in for drug-delivery/controlled release applications since it would be desirable for the material to maintain its integrity during the release time of the drug and then after release for the material to degrade and be flushed from the body.

Whereas particular embodiments of the invention have been described herein for purposes of illustration, it will be evident to those skilled in the art that numerous variations of the details may be made without departing from the invention as set forth in the appended claims.

1. A composition comprising a biomolecule-containing metal-organic framework having pores structured therein, said framework comprising a metal component and a biomolecule component, said pores capable to adsorb material therein.

2. The composition of claim 1, wherein the metal component is selected from the group consisting of metal cluster, metal ion or combination thereof.

3. The composition of claim 1, wherein the metal component is selected from the group consisting of Cu, Ti, Cr, Fe, Ni, Mn, Co, Zn, GaN, BN, Si₃N₄, TiN, TiC, TiB₂ GaAs, GaP, InP, AlP, and salts and mixtures thereof.

4. The composition of claim 1, wherein the biomolecule comprises a nucleobase.

5. The composition of claim 4, wherein the nucleobase is selected from the group consisting of adenine, guanine, cytosine, thymine, uracil and mixtures thereof.

6. The composition of claim 1, wherein the biomolecule is biologically compatible with the metal component.

7. The composition of claim 1, further comprising an organic component.

8. The composition of claim 7, wherein the organic component comprises an organic ligand.

9. The composition of claim 8, wherein the organic ligand comprises a multitopic material.

10. The composition of claim 8, wherein the organic ligand is selected from the group consisting of dicarboxylate ligands, tricarboxylate ligands, tetracarboxylate ligands, other multicarboxylated ligands, dipyridyl ligands, tripyridyl ligands, tetrapyridyl ligands, other multipyridal ligands, dicyano ligands, tricyano ligands, tetracyano ligands, other multicyano ligands, diphosphonate ligands, triphosphonate ligands, tetraphosphonate ligands, other multiphosphonate ligands, dihydroxyl ligands, trihydroxyl ligands, tetrahydroxyl ligands, other multihydroxyl ligands, disulfonate ligands, trisulfonate ligands, tetrasulfonate ligands, other multisulfonate ligands, diimidazolate ligands, triimidazolate ligands, tetraimidazolate ligands, other multiimidazolate, ligands, ditriazolate ligands, tritriazolate ligands, tetratriazolate ligands, other multitriazolate ligands, and mixtures and combinations thereof.

11. The composition of claim 1, wherein the framework is selected from the group of structures consisting of macrocyclic structures, crystalline structures, polyhedra, extended framework structures and combinations thereof.

12. The composition of claim 1, wherein the framework comprises a plurality of units connected by a linker.

13. The composition of claim 12, wherein the linker is selected from the group consisting of 1,4-benzene dicarboxylate; 2,6-naphthalene dicarboxylate; 4,4′-biphenyl dicarboxylate; 4,4″-terphenyl dicarboxylate; 4,4′-[(2,5-dimethoxy-1,4-phenylene)di-2,1-ethenediyl]bis-benzoic acid; 1,3,5-benzene tricarboxylate; 4,4′,4,4″-benzene-1,3,5-triyl-tribenzoate; 4,4′,4″-[benzene-1,3,5-triyl-tris(ethyne-2,1-diyl)]tribenzoate; 4,4′,4,4″-[benzene-1,3,5-triyl-tris(benzene-4,1-diyl)]tribenzoate; and mixtures thereof.

14. The composition of claim 1, wherein the metal component comprises zinc salt.

15. The composition of claim 5, wherein the nucleobase comprises adenine.

16. The composition of claim 1, wherein the material is selected from the group consisting of gas, drug, protein, polymer, and combinations thereof.

17. The composition of claim 16, wherein the gas is selected from the group consisting of carbon dioxide, hydrogen, nitrogen and mixtures thereof.

18. The composition of claim 16, wherein the material is drug and the framework is capable of a controlled release of said drug from said pores.

19. The composition of claim 1, wherein the pores can be modified by cation exchange of cations in the pores with different cations.

20. The composition of claim 1, wherein the biomolecule-containing metal-organic framework has a BET surface area from about 1000 to about 4000 m²/g.

21. The composition of claim 1, wherein the biomolecule-containing metal-organic framework has a pore volume from about 2 to about 6 cm³/g.

22. The composition of claim 1, wherein the biomolecule-containing metal-organic framework has a pore volume of about 4.2 cm³/g.

23. A method for preparing a biomolecule-containing metal-organic framework having pores structured therein, said pores capable to adsorb materials therein, the method comprising reacting a metal component and a biomolecule component.

24. A method for storing and controllably releasing drug material comprising the composition of claim 1.

25. A method for capturing and storing carbon dioxide material comprising the composition of claim 1.
